(19) United States
(12) Patent Application Publication
(10) Pub. No.: US 2024/0325039 A1
(43) Pub. Date: Oct. 3, 2024

(54) Nasal Scraper

(71) Applicant: Pietro Maniaci, Grosse Pointe Woods, MI (US)

(72) Inventor: Pietro Maniaci, Grosse Pointe Woods, MI (US)

(21) Appl. No.: 18/623,112

(22) Filed: Apr. 1, 2024

Related U.S. Application Data
(60) Provisional application No. 63/456,546, filed on Apr. 3, 2023.

(51) Int. Cl. A61B 17/24

(52) U.S. Cl. CPC A61B 17/24; A61B 2017/246

(57) ABSTRACT

A device for removing a substance from a nasal cavity, the device having a handle; a working tip; and a stopper connected to the working tip, the stopper being operative to limit a depth of insertion of the working tip into the nasal cavity; the distal end of the handle has a working tip engagement feature configured to receive a handle engagement feature of the working tip to connect the working tip and the handle; the working tip includes a stopper engagement section and the stopper includes a working tip engagement section; and the stopper engagement section and the working tip engagement section include a corresponding helical threaded connection so that the stopper is rotatable about the working tip to increase and decrease a distance between the working tip and the stopper.

### CROSS REFERENCE TO RELATED APPLICATIONS

This application claims priority to U.S. 63/456,546 filed on Apr. 3, 2023, the entirety of which is hereby incorporated by reference herein for all purposes.

### FIELD

These teachings relate to a device and method for removing matter from inside a nasal cavity.

### BACKGROUND

A person may be tempted to use a nontraditional method and/or device for removing matter from inside a nasal cavity. For example, a person may use a finger, tissue, or a cotton swab to remove or dislodge irritating matter from within their nose. Additionally, or alternatively, an individual or medical professional may seek to extract matter from within a nasal cavity to test for certain medical conditions or illnesses. However, a danger of using a cotton swab or other device is that the device may be inserted too deeply into the nasal cavity thereby causing pain and or irritation. It would be desirable to have a device that has a feature for limiting the depth in which it can be inserted into the nasal cavity, thereby reducing or preventing injury and or discomfort.

### SUMMARY

These teachings relate to a device and method for cleaning or removing matter from a nasal cavity. The device includes a handle with a distal end and a proximal end; a working tip located at one or both of the ends of the handle, and configured to be inserted into the nostril; and a stopper connected to the handle and disposed proximal of the working tip, the stopper including a size that is greater than an opening to the nostril, the stopper being configured to limit a depth of insertion of the working tip into the nasal cavity; and, advantageously, a distance between the stopper and working tip may be adjustable.

While the teachings herein focus on the device and method being configured for human nasal applications, it is understood that these teachings may also find use in other human and/or animal orifice applications including ear, nose, throat, and other orifices.

### BRIEF DESCRIPTION OF THE DRAWINGS

FIG. 1 is a side view of the device.

FIG. 2 illustrates examples of a shape of the stopper.

FIG. 3 illustrates examples of the working tip.

FIG. 4 is a side view of the device.

FIG. 5A illustrates a step of using the device.

FIG. 5B illustrates a step of using the device.

FIG. 5C illustrates a step of using the device.

FIG. 5D illustrates a step of using the device.

FIG. 6 is a partial side view of the device.

FIG. 7A is a partial side view of the device.

FIG. 7B is a partial side view of the device.

FIG. 8 is a partial side view of the device.

FIG. 9 is a perspective view of a kit, that includes the device and various exemplary working tips.

FIG. 10 is a perspective view of the handle.

FIG. 11 is a perspective view of the stopper.

FIG. 12A is a cross-sectional view of the device.

FIG. 12B is a cross-sectional view of the device.

### DETAILED DESCRIPTION

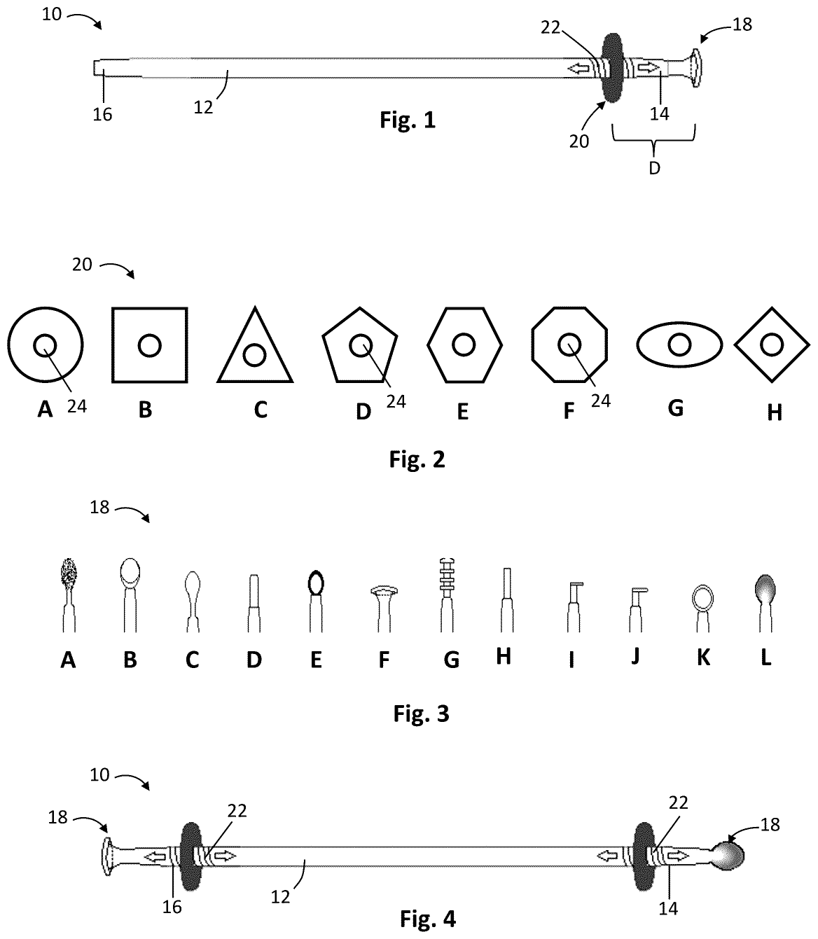

FIG. 1 illustrates a device 10 according to these teachings. The device 10 includes a handle 12 having a distal end 14 and an opposing proximal end 16. A working tip 18 may be located at or connected to the distal end 14. As will be discussed further below, the working tip 18 and/or at least part of the handle 12 (depending on a position of a stopper 20) may be configured to be inserted into a nasal cavity.

A stopper 20 may be connected to the handle 12. The stopper 20 may be connected to the handle 12 in a location or region between the distal end 14 and the opposing proximal end 16. The stopper 20 may be located closer to the distal end 14 than the proximal end 16. As will be discussed further below, the position of the stopper 20 relative to the proximal and distal ends is adjustable.

The stopper 20 may be located proximal to the working tip 18 of the device 10. The stopper 20 may include a size and or shape that is greater than a typical size or opening to the nostril or other orifice. The stopper 20 may be configured to limit a depth of insertion of the working tip 18 into the nasal cavity. A distance D defined between the stopper 20 and the working tip 18 may be adjustable.

The handle 12 may include a stopper engagement section 22 along its length. The stopper engagement section 22 may be located only partially along the length of the handle 12, for example in a region or location closer to the distal end 14 of the handle 12. However, in some configurations, the stopper engagement section 22 may instead extend more than partially or even along an entire length of the handle 12 between the ends 14, 16. The stopper engagement section 22 may be configured to engage the stopper 20. In some configurations, the stopper engagement section 22 may also function as a gripping surface for a user to grasp onto without the device 10 or handle 12 slipping from the user hand. The stopper engagement section 22 may be a helical thread, as illustrated in FIG. 1. In some configurations, the stopper engagement section 22 may also or may instead be located on the working tip 18 of the device.

The stopper 20 may have virtually any shape. For example, with continued reference to FIG. 2, the stopper 20 may have a circular shape (example A); a square shape (example B); a triangle shape (example C); a pentagon shape (example D); a hexagon shape (example E); an octagon shape (example F); an oval shape (example G); or a diamond shape (example H). Other shapes are within the scope of this disclosure, and may include a star shape, a teardrop shape, an irregular shape, etc. or a combination thereof. The stopper 20 may also resemble a logo, face, or character, such as a smiley face, or a known children's television or movie character.

The stopper 20 may be made from a suitable material, such as a compressible or flexible material. A rubber or soft, compressible, or flexible material may be used. For example, the stopper 20 may comprise a Shore A silicone material. However, in some configurations a rigid and non-pliable material may be used. For example, a plastic, metal, composite, or a 3D printing material may be used.

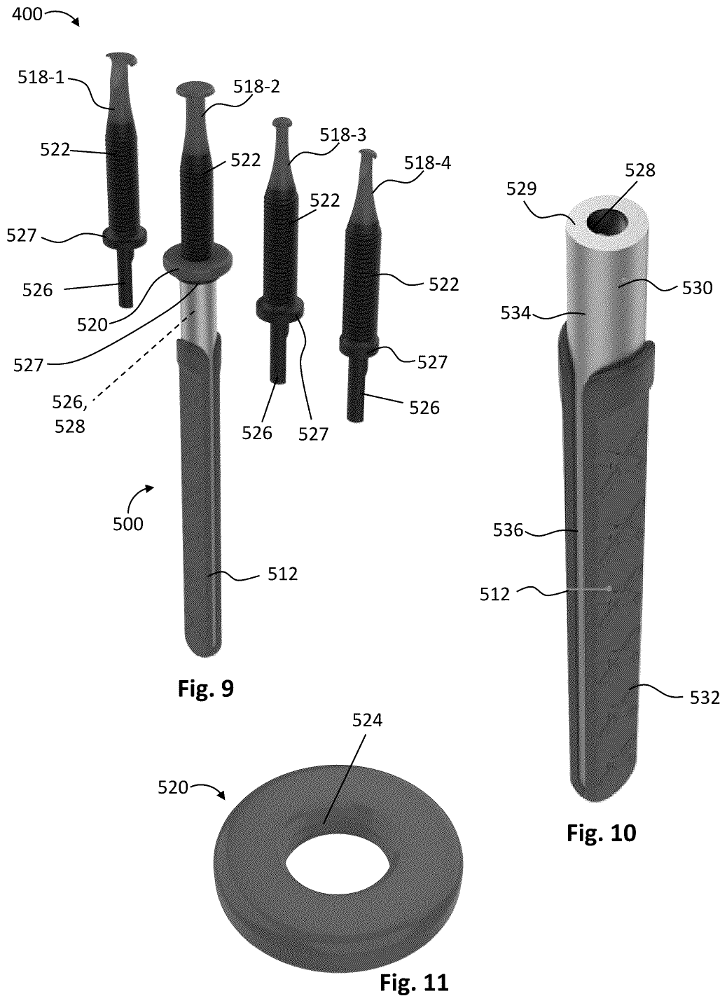

The stopper 20 may include a handle engagement section 24. The handle engagement section 24 may function to connect or attach the stopper 20 to the handle 12, the stopper engagement section 22, or both. The handle engagement section 24 may also be referred to generally as an engagement section 24, especially if the engagement section 24 is configured to connect the stopper 20 to the working tip part as illustrated and described for example in the later FIGS. 9-12B.

The handle engagement section 24 may include a connecting structure that can mate with or connect to the stopper engagement section 22. For example, the stopper engagement section 22 may be a helical thread that is configured to mate with a mating helical thread in the handle engagement section 24. Other mating structures that may be used with or in place of the threaded/helical connection between the stopper engagement section 22 and the handle engagement section 24 may include a magnetic connection, a biasable projection and orifice connection, a friction engagement that must be overcome to move the stopper along the stopper engagement section 22, a spring button that engages an opening, or a combination thereof. The stopper engagement section 22 may be located at a substantially central region of the stopper 20. However, stopper engagement sections 22 that are offset from a central axis of the stopper 20 are also contemplated. The stopper 20 may also be configured to be removable. The removability of the stopper 20 may allow for another stopper to be installed in its place. For example, in the event the stopper becomes damaged or unfit for its intended purpose, the old stopper may be removed and a new stopper may be installed. Or a second stopper or a stopper having a different shape or configuration may be installed. In some configurations, the stopper may be removed and the device may be used without the stopper if so desired by a user or medical professional. The stopper may be removed for cleaning the stopper, the handle, the working tip, or a combination thereof.

The handle engagement section 24 may be made of the same material as the remainder of the body of the stopper 20 or from a different material. For example, the handle engagement section 24 may be made of a rigid material and the rest of the stopper 20 may be made of a soft compressible material. The handle engagement section 24 may be a rigid metal or aluminum material that is insert molded or over molded with the rest of the stopper material that may be a plastic, rubber, or silicone material. This may ensure that the integrity of the handle engagement section 24 and or the helical threads are not compromised during use. The stopper engagement section 22 may be made of the same material as the handle 12 or from a different material. For example, the stopper engagement section 22 may be insert molded or over molded with the handle 12.

The working tip 18 of the device 10 may be located at and/or connected to the distal end 14 of the handle 12. In some configurations, the working tip 18 may be removably attached to the handle 12. This is exemplified in the examples described at FIGS. 9, 10, 12A-12B. Removably attached means that the working tip 18 can be separated from the handle 12 without damaging one or both of the working tip 18 and the handle 12. A removable or replaceable working tip 18 may be advantageous in that the working tip 18 may be replaced or substituted with a different working tip 18, based on user preference, if a tip 18 is damaged or worn out, and/or for the specific intended use. For example, the working tip 18 may be connected to the handle 12 via a suitable method or mechanism such as, for example, a threaded engagement (screw-on and screw-off), a push or friction fit, a magnet, fastener such as an adhesive, a quick connect fitting, spring loaded, etc. In some configurations, the working tip 18 may be permanently attached to the distal end 14 (and/or proximal end) of the handle 12. Permanently attached means that the working tip 18 cannot be separated from the handle 12 without damaging one or both of the working tip 18 and the handle 12. For example, the working tip 18 may be integrally formed with the handle 12 (in one or more manufacturing processes from the same material), or the working tip 18 may be mechanically attached to the handle 12 via one or more adhesives and/or fasteners.

FIG. 3 illustrates several examples of a working tip 18. Example A illustrates a working tip 18 that is a swab or other soft cloth or cotton structure. Examples B and L illustrate various working tips 18 that are like a spoon and may be slightly more bowled like that of an ice cream scoop or a pumpkin scraper. The spoon/scooped end in Examples B and L may be relatively thin, though dull at their edges and not sharp enough to cut or scrape the nasal mucosa, rather only sharp enough to safely and effectively scrape debris. The size of the edge of the spoon/scoop/scraper at this end is to be smaller than the opening of the user's nostrils to allow for easy access in through the openings of the nostrils. The product may come in several sizes, some larger than others to fit different sized nasal passages. The working tip 18 illustrated in Example C includes a cotton-tip ball which may be commonly known as sold by Q-TIPS®. The working tip 18 illustrated in Example D may be a rounded at the corner, straight edge tip that may be used to scrape out orifices with rounded walls or more safely scrape out orifices with straight walls. The working tip 18 illustrated in Examples E and K may be a loop curette that may help to scrape or loosen debris within the car canal or nasal passage without also necessarily effectively removing debris. Example F illustrates a 360 Degree working tip 18 that may be thin, still dull at its edge but not thin enough or sharp enough to cut the nasal mucosa, only to allow for adequate removal of dry or moist debris. The overall size/circumference of this tip may be smaller than the opening of the user's nostrils to allow for easy access in through the nostrils. The shape of this tip looks like that of a fountain, with a scraper that is a complete circle, round by 360 degrees to allow for easy removal of dry mucus from all walls of the nasal mucosa without having to twist the device itself. Example G illustrates a working tip 18 having a plurality of rings or a helically wound wire. Example H illustrates a straight edge tip that may be used to scrape out orifices or other cavities with straight interior walls i.e. straight pipes. Examples I and J illustrate a working tip 18 having a squared Edge 90-degree and a rounded Edge 90-degree feature, respectively.

One or more of the working tips 18 illustrated and/or described herein may be used for removing matter or samples from a nostril or other orifice of the body. The samples may be used to test for various bacteria or contaminants that may have found their way into the human body. One or more of the working tips 18 may be used for sample collection to diagnose certain illnesses and/or other health conditions. One or more of the working tips may be configured to scrape or otherwise remove matter from the body. In other configurations, the one or more working tips may be used for collecting samples or removing matter from regions of the body that are not necessarily included in an orifice or opening. For example, one or more of the open working tips may be used for scraping or collecting a matter from an exposed region of the body. One or more of the working tips 18 may be configured for massaging and/or scratching “an itch” within the nostril or orifice. In certain configurations, the orifice may be a naturally occurring opening or cavity (e.g., car, nose, throat, vagina, anus, etc.). In other configurations, the orifice may be a non-naturally occurring orifice, such as in the case of a surgical procedure where orifice or cavity is created for various reasons, for example to extract matter, a sample, or an otherwise foreign substance. In such a procedure, the device and stopper may be used to, advantageously and safely, prevent the working tip from penetrating too deeply into the orifice or cavity.

Other working tip 18 designs may include: a flashlight, testing swab for lab samples of bacteria or virus, an expandable and/or constable design, a grasping or clamping tool such as pliers, tweezers, or basket, a cutting tool such as a razor or scalpel, a camera for inspecting the orifice, a hair cutting tool such as a nose hair trimmer or scissors, a vibrating or massaging device, or a combination thereof. In some configurations, the working tip 18 may be connected to a vacuum source so that the working tip 18 may be configured to suction matter or debris. In some configurations, the working tip 18 may be connected to a source of air so that pressurized air may be expelled from the working tip. In some configurations, the working tip 18 may be connected to a source of water or fluid so that a pressurized water or other fluid such as saline may be expelled from the working tip to rinse or wash an affected area or orifice.

FIG. 4 illustrates a device 10. The device 10 may be similar in structure and/or function to the device 10 illustrated and described above at FIGS. 1-3. In FIG. 4, the device 10 includes a working tip 18 at both of the distal 14 and proximal ends 16. A stopper 20 may engage a stopper engagement section 22 on the handle 12 and may be moved along the length of the stopper engagement section 22 to change or adjust the distance between the working tip 18 and the stopper to prevent over-insertion of the working tip 18 into an orifice.

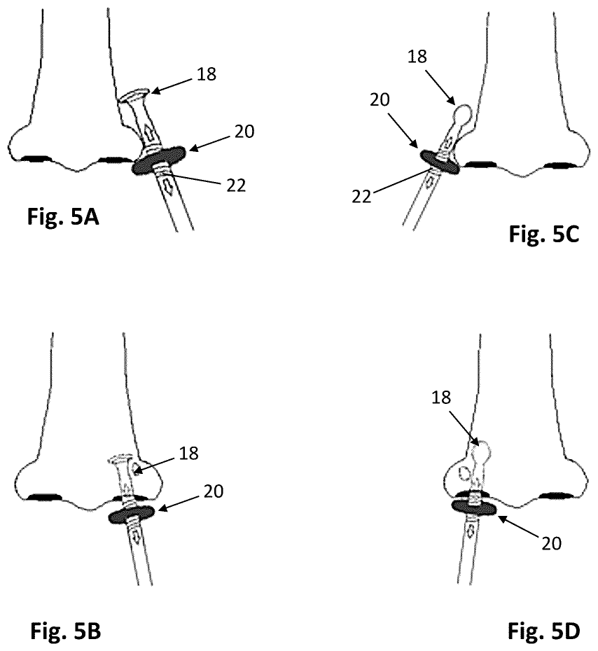

FIGS. 5A-5D illustrate an exemplary method for using the device according to these teachings.

In FIG. 5A, a user may move and set the position of the stopper 20 relative to the working tip 18 by resting the working tip 18 on the outside of the user's nose with the stopper 20 placed just below the nostril and optionally touching the nostril to simulate insertion and adjust to a desired level of safety prior to insertion. The distance between the stopper 20 and the working tip 18 may be adjusted by rotating the stopper 20 relative to the handle 12 (and/or rotating the handle 12 relative to the stopper 20) to move the stopper 20 along a length of the handle 12 and/or along the length of the stopper engagement section 22. The stopper 20 may be rotated in a clockwise or counter-clockwise direction to adjust the position of the stopper 20 relative to the working tip 18.

In FIG. 5B, the user may then insert the working tip 18 into the nasal cavity. The user may then gently scrape the inner walls of the nose to remove or dislodge any matter. Afterwards, the device and working tip 18 may be withdrawn from inside the nasal cavity and cleaned. The process of FIG. 5B may be repeated until a sufficient amount of the substances are removed or are loosened from the nose.

In FIG. 5C, a user may repeat the step of FIG. 5A for the other nostril. Because the depth or size of the nostril and/or nasal cavity may be different (i.e., larger or smaller or wider or narrower) then the size of the nostril and/or nasal cavity in FIG. 5A, a user may wish to set the position of the stopper 20 relative to the working tip 18 to ensure the working tip 18 is not over inserted beyond a comfortable point. In FIG. 5D, the user may then perform similar steps as in FIG. 5B, to loosen and or remove material from inside the nasal cavity.

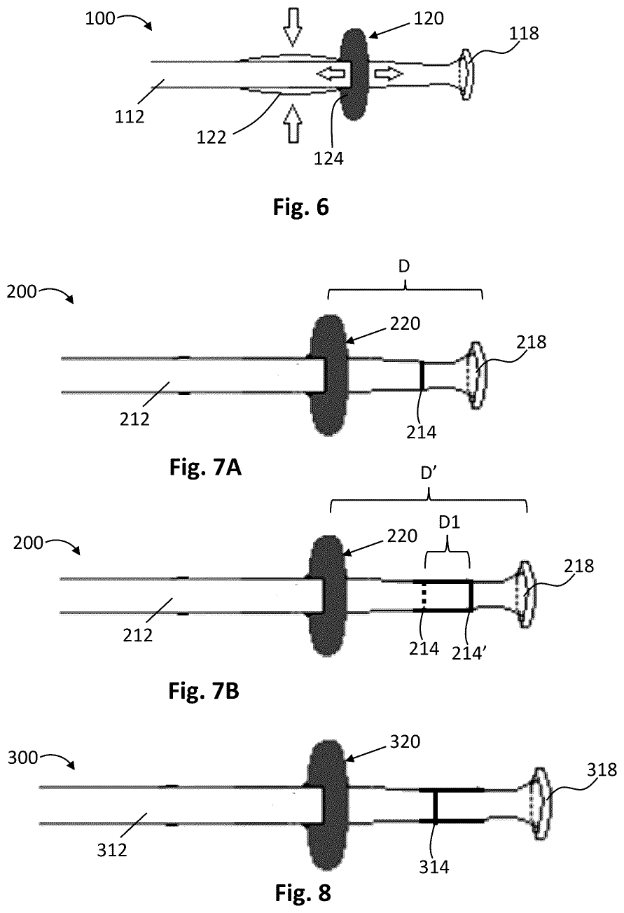

FIG. 6 illustrates another device 100 that may be substantially similar to the device illustrated and described above at reference numeral 10. Therefore, only the differences will be described hereafter in the interest of brevity. It is understood that any teachings related to any of devices disclosed herein may be added or combined with the device 100 and vice versa. The difference in reference numerals is only for ease of describing the features of the device.

The device 100 may include a stopper engagement section 122 that is configured to co-act with the handle engagement section 124 of the stopper 120. The stopper engagement section 122 may comprise a compressible, deflectable, de-flatable, biasable, and/or squeezable section. Prior to adjusting the position of the stopper 120 relative to the working tip 118, user may squeeze the handle engagement section 122 thereby compressing or reducing the size of the handle engagement section 122. This may reduce the size of the stopper engagement section 122 thereby allowing the stopper 120 to be moved along a length of the handle 112 in order to change or adjust the distance and position of the stopper 120 relative to the working tip 118. After the stopper 120 is set to a preferred location, the user may release the pressure application on the stopper engagement section 122, which may cause the stopper engagement section 122 to expand and return to its size, thereby preventing the stopper 120 from any further movement. The stopper engagement section 122 may be located at only one end of the handle 112 or at both ends. The stopper engagement section 122 may be located on the handle 112 or may be part of the working tip 118 that is connected to the handle 112.

FIGS. 7A and 7B illustrate another device 200 that may be substantially similar to the devices illustrated and described above at reference numerals 10 and 100. Therefore, only the differences will be described hereafter in the interest of brevity. Again, it is understood that any teachings related to any of devices disclosed herein may be added or combined with the device 200 and vice versa. The difference in reference numerals is only for case of describing the features of the device.

The device 200 has a handle 212 that may be configured to change length. In this configuration, a position of the stopper 220 may be fixed or at least partially restricted or prevented from moving along a length of the handle 212. To change the relative position or distance between the stopper 220 and the working tip 218, the distal end 214 of the handle 212 may be elongated or extended. For example, a telescoping structure may be incorporated into the design of the device 200 that allows for the overall length of the handle 212 to be extended when pulled and then contracted when compressed. For example, the distance D between the stopper 220 and the working tip 218 (FIG. 7A) may be increased to DI (FIG. 7B) by extending or pulling the distal end 214 of the handle 212 from its position in FIG. 7A to its new position 214′ in FIG. 7B. It is envisioned that this telescoping structure may resemble a radio antenna. Of course, in another configuration, the stopper 220 may be adjusted along the length of the handle via engagement and cooperation of a stopper engagement section and a handle engagement section.

Additionally, or alternatively, it may be envisioned that the distal end 214 of the handle 212 or device 200 may be moved by turning and causing the distal end to 214 to be moved or displaced relative to the stopper 220 along an internal threaded and/or telescoping structure. This will result in the overall distance between the working tip 218 and the stopper 220 to change positions.

FIG. 8 illustrates another device 300 that may be substantially similar to the device illustrated and described above. Therefore, only the differences will be described hereafter in the interest of brevity. Again, it is understood that any teachings related to any of devices disclosed herein may be added or combined with the device 300 and vice versa. The difference in reference numerals is only for ease of describing the features of the device.

In FIG. 8, rather than the distal end 314 of the handle 312 being movable or displaceable relative to the stopper 320 like the example illustrated and described above at FIGS. 7A an 7B, the working tip 318 may be movable or displaceable relative to the stopper 320 to change or adjust the distance or position between the stopper 320 and the working tip 318. A linear telescoping or threaded structure is also envisioned for this embodiment.

FIG. 9 illustrates a kit 400 that may include a device 500 and one or a plurality of working tips 518-1, 518-2, 518-3, 518-4, etc. Any number of working tips may be included in the kit 400, including only one working tip, or some or all of the working tips illustrated and/or described herein.

With additional reference to FIG. 10, the handle 512 may be an elongated member that has a generally elongated, cylindrical shape with a round cross-section. The handle 512 may include an inner handle part 530 and an outer handle part 532. The outer handle part 532 may be a grip or over mold on the inner handle part 530 and configured to provide a grip or tactile feel for the handle or device. The outer handle part 532 may only partially cover the inner handle part 530. For example, the distal end region 534 of the inner handle part 530 may be open, exposed, or free from being covered by the outer handle part 532. For example, at least a portion 536 of the length of the inner handle part 530 may be free from being covered by the outer handle part 532. The outer handle part 532 may be round like the inner handle part, or it may be that the outer handle part is substantially flat along its length. In some configurations, the outer handle part 532 may cover an entirety of the inner handle part 530 such that the inner handle part 530 is not exposed.

In some configurations, the outer handle part 532 may be omitted. In other configurations, the outer handle part 532 may be separable from the inner handle part 530. In such a removable configuration, the outer handle part 532 may function like a pouch or cover. In some configurations, the outer handle part 532 may cover or protect the working tip 518 from damage or germs when not in use. In some configurations, the outer handle part 532 may be permanently attached to the inner handle part 530 such that removable or separation of the outer handle part 532 may cause damage to one or both of the handle parts 530, 532.

The handle 512 may include a working tip engagement feature 528 at one or both of its proximal and distal ends. The working tip engagement feature 528 may be a bore or opening at one or both ends of the handle 512. The working tip engagement feature 528 may be configured to receive at least part of a handle engagement feature 526 of any of the working tips disclosed herein. A handle engagement feature 526 of the working tip 518 may be an extension or projection extending from the working tip 518.

The working tip 518 may be connected to the handle by inserting the handle engagement feature 526 into the working tip engagement feature 528 of the handle. The fit between the two parts 518, 526 may be a friction fit (i.e., a substantially line-to-line fit). In other configurations, the two parts 518, 526 may threadably engage one another; magnets may be used to create a lock between the two parts 518, 526. Preferably, the fit between the two engagement features 526, 528 is such that the working tip 518 is restricted or prevented from freely moving or rotating relative to the handle.

The working tip 518 has a stopper engagement section 522 that is configured to co-act with the engagement section 524 on the stopper 520 (see FIG. 11). The engagement sections 522, 524 are threaded engagements so that a distance between the stopper 520 and the working tip 518 can be adjusted (increased and/or decreased) by rotating or spinning the stopper 520 relative to the handle 512 or vice versa.

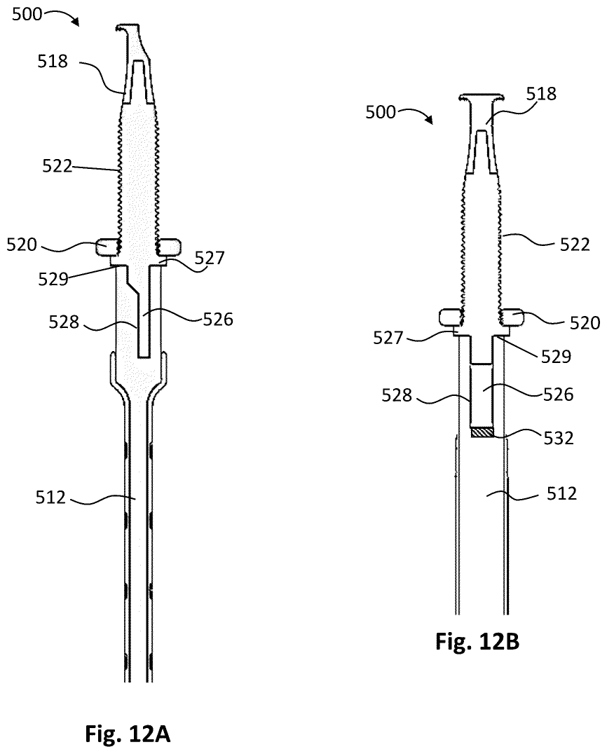

With additional reference to FIGS. 12A and 12B, which are cross-sectional views of the device 500, the handle engagement feature 526 of a working tip 518 is shown engaging the working tip engagement feature 528 of the handle 512. The engagement may be a press fit or friction fit. In other configurations, one or more magnets 532 may be present at the engagement of the features 526, 528 to facilitate a secure or robust connection between the two elements. In other configurations, the connection between 526, 528 may be with helical threading in which the user inserts the working tip 518 into the handle 512 by twisting the working tip 518 like a bolt into a nut. The fit between the two engagement features 526, 528 is such that the working tip 518 is restricted or prevented from freely moving or rotating relative to the handle. For example, the fit may include an offset or planar surfaces that restrict or prevent rotation of the working tip 518 relative to the handle 512.

The working tip 518 has a shoulder or flange 527. The shoulder or flange 527 may be located in a region of the working tip between the stopper engagement section 522 and the handle engagement section 526. The shoulder or flange 527 may extend at least partially, or entirely, around the working tip 518 or features thereof. Upon insertion of the handle engagement section 526 into the working tip engagement feature 528, the shoulder or flange 527 may be configured to contact or butt up against the face or the distal end 529 of the handle 512 to provide a positive stop and/or to prevent further insertion of the working tip 518 into the handle 512. Therefore, the shoulder or flange 527 may co-act with the distal end 529 of the handle 512 to prevent over insertion of the working tip 518 into the handle 512. The shoulder or flange 527 may also function as a positive stop to prevent the stopper 520 from disengaging or being threaded off from the distal end of the stopper engagement section 522 during retraction of the stopper if the stopper 520 is unscrewed from the section 522 too far in that direction. The shoulder or flange 527 contacting the distal end or face 529 of the handle can be appreciated when reviewing FIGS. 12A and 12B. The stopper 520 at its bottom-most position and contacting the shoulder or flange 527 can also be seen in FIGS. 12A and 12B.

The shoulder or flange 527 may have an overall size or diameter or cross section that is greater than a size or diameter or cross section of the handle 512 and/or the inner handle part 530. The stopper 520 may have a diameter or diameter or cross section that is larger than the shoulder 527.

FIGS. 12A and 12B may also contemplate that the working tip 518 may be removably connected to the stopper engagement section 522. Such a connection may facilitate only changing out the actual working tip 518, while the stopper engagement section 522 remains fixedly or removably connected to the handle 512. The connection between the working tip 518 and the stopper engagement section 522 may be a press fit, threaded, or friction fit connection, or like any of the other connections illustrated and/or described herein.

The device according to these teachings may be referred to as a nasal scraper, a scraper, a pick, a pick stick, a booger buddy, a booger scraper, a pic, a nosepick, a Nose Pick, or any combination thereof.

The handle, working tip, stopper, and any of the other elements disclosed herein may be made from any suitable material and/or manufacturing process. For example, the handle, working tip, stopper, or combination thereof may be made from a plastic, polypropylene, silicone, a medical grade material, a biodegradable material, a metal material, a 3D printed material, a composite material, or combination thereof. Any of the elements disclosed herein may be made via a suitable manufacturing process such as plastic injection molding, extrusion, 3D printing, or a combination thereof.

The handle may be substantially elongated between its proximal and distal ends. The handle may be substantially rigid between its proximal and distal ends thereby restricting or preventing the handle from bending. This may help ensure the structural integrity of the handle is not compromised during a procedure. However, in some configurations, the handle may be curved or have regions that are not substantially linear. In such a configuration, the handle may be designed in order to help facilitate improved gripping or handling by a user and or improved positioning or access for the working tip to perform its desired function. In some configurations the handle may be bendable. In such a configuration, a bendable handle may also have shaped memory which will allow a user to fine tune the geometry of the handle to provide the best fit for the user and positioning of the handle and working end in its intended location. The handle may be of any suitable length. For example, the handle may have a length on the order of about two inches or more, 3 inches or more, 4 inches or more, 5 inches or more, 6 inches or more, 7 inches or more, 8 inches or more, 9 inches or more, 10 inches or more, 11 inches or more, or 12 inches or more. A cross section of the handle may be round, oval, square, triangular, have one or more planar walls or sides, have one or more curved walls or sides, or any combination thereof. The handle may include one or more gripping features or services such as one or more bumps, grooves, depressions, indentations, bulges, naturals, or combination thereof. Such features may restrict or prevent the handle from slipping during use.

The working tip and/or features of the working tip may be substantially linear. However, in some configurations one or more regions of the working tip may be curved or bendable in order to help facilitate an improved manner of scraping or removing the substance from the orifice or opening. The working tips may be for one time use which means that after a use it is desired or recommended to dispose of the working tip. In other configurations, the working tip may be reusable. For example, the working tip may be sterilized or put into an autoclave in order to ensure a clean and sterile working tip for future uses. The working tip may be made from a shape memory material, which may allow a user to bend or manipulate the shape or geometry of the working tip prior to or during use.

The stopper has an engagement section that is configured to mate with and/or engage a corresponding engagement section, which can be provided on the handle, on the working tip, or both. For example, in some exemplary FIGS. 1, 4, 6, etc., the corresponding engagement section that the stopper engages is provided on the handle. In other exemplary figures like in FIG. 9, 12A-B, the corresponding engagement section that the stopper engages is provided on the working tip. It is therefore envisioned that depending on the specific configuration of the device, the engagement section of the stopper may engage a corresponding engagement section on the handle, working tip, or both.

The foregoing description applies to all examples. That is, any of the working tips may be used with any of the devices disclosed herein. Any of the stopper engagement sections may be used with any of the devices, stoppers, and working tips disclosed herein. One or more features of the device, stopper engagement sections, stoppers or working tips may be combined with one or more other devices, stopper engagement sections, stoppers, working tips, and vice versa. Any features disclosed herein may be omitted or duplicated.

The explanations and illustrations presented herein are intended to acquaint others skilled in the art with the invention, its principles, and its practical application. The above description is intended to be illustrative and not restrictive. Those skilled in the art may adapt and apply the invention in its numerous forms, as may be best suited to the requirements of a particular use.

Accordingly, the specific embodiments of the present invention as set forth are not intended as being exhaustive or limiting of the teachings. The scope of the teachings should, therefore, be determined not with reference to this description, but should instead be determined with reference to the appended claims, along with the full scope of equivalents to which such claims are entitled. The omission in the following claims of any aspect of subject matter that is disclosed herein is not a disclaimer of such subject matter, nor should it be regarded that the inventors did not consider such subject matter to be part of the disclosed inventive subject matter.

Plural elements or steps can be provided by a single integrated element or step. Alternatively, a single element or step might be divided into separate plural elements or steps.

The disclosure of “a” or “one” to describe an element or step is not intended to foreclose additional elements or steps. For example, disclosure of “a handle” does not limit the teachings to a single handle. Instead, for example, disclosure of “a handle” may include “one or more handles.”

While the terms first, second, third, etc., may be used herein to describe various elements, components, regions, layers and/or sections, these elements, components, regions, layers and/or sections should not be limited by these terms. These terms may be used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The invention illustratively disclosed herein suitably may be practiced in the absence of any element which is not specifically disclosed herein.

Any of the elements, components, regions, layers and/or sections disclosed herein are not necessarily limited to a single embodiment. Instead, any of the elements, components, regions, layers and/or sections disclosed herein may be substituted, combined, and/or modified with any of the elements, components, regions, layers and/or sections disclosed herein to form one or more embodiments that may not be specifically illustrated or described herein.

The disclosures of all articles and references, including patent applications and publications, testing specifications, are incorporated by reference for all purposes. Other combinations are also possible as will be gleaned from the following claims, which are also hereby incorporated by reference into this written description.

1. A device configured for removing a substance from a nasal cavity, comprising:
a handle having a distal end and a proximal end, the distal end comprising a working tip engagement feature;
a working tip configured to be inserted into the nasal cavity, the working tip comprising a stopper engagement section, a handle engagement feature, and a circumferential shoulder located between the stopper engagement section and the handle engagement feature; and
a stopper movably connected to the working tip, the stopper including a size that is greater than an opening to the nasal cavity, the stopper being operative to limit a depth of insertion of the working tip into the nasal cavity, the stopper comprising a working tip engagement section;
wherein the working tip engagement feature is configured to receive the handle engagement feature of the working tip to connect the handle and the working tip, wherein the circumferential shoulder of the working tip is configured to contact a distal end of the handle after the working tip is connected to the handle;
wherein the working tip engagement section of the stopper is configured to engage the stopper engagement section of the working tip to connect the stopper to the working tip; and
wherein the stopper engagement section and the working tip engagement section include a corresponding helical threaded connection so that the stopper is rotatable about the working tip to increase and decrease a distance between the working tip and the stopper.

2. The device according to claim 1, wherein handle engagement feature is a projection that extends from a proximal end of the working tip, and the working tip engagement feature is a bore defined in a distal end of the handle, and wherein the circumferential shoulder of the working tip is configured to prevent the stopper from disengaging the stopper engagement section during retraction of the stopper.

3. The device according to claim 2, wherein the handle has a substantially cylindrical shape, the shoulder has a diameter that is greater than a diameter of the handle, and the stopper having a diameter that is larger than the shoulder.

4. The device according to claim 3, wherein the handle comprises an inner handle part and an outer handle part.

5. The device according to claim 4, wherein the outer handle part only partially covers the inner handle part.

6. The device according to claim 5, wherein the distal end of the inner handle part is free from being covered by the outer handle part.

7. The device according to claim 6, wherein at least a portion of the length of the inner handle part is free from being covered by the outer handle part.

8. The device according to claim 7, wherein outer handle part is substantially flat along its length.

9. The device according to claim 8, wherein the stopper comprises an inner member and an outer member, wherein the inner member is made of a substantially rigid member and the outer member is made of a compressible or flexible material.

10. The device according to claim 9, wherein the inner member comprises an insert having a threaded part.

11. The device according to claim 1, wherein the working tip comprises a spoon, scraper, loop curette, flashlight, swab, rounded edge, square or linear edge, cotton tip, suction device, or a combination thereof.

12. A method comprising:
inserting the working tip into an opening defined at a distal end of a handle of the device according to claim 1;
adjusting a distance between the stopper and the working tip by rotating the stopper about the stopper engagement feature of the working tip;
inserting the working tip of the device into the nasal cavity or the other orifice until the stopper contacts the opening into the nasal cavity or the opening to the other orifice.
